(19) United States
(12) Patent Application Publication
(10) Pub. No.: US 2020/0030000 A1
(43) Pub. Date: Jan. 30, 2020

(54) APPARATUS FOR PUNCTURING MAXILLARY SINUS

(71) Applicants: Yeong Seok YUN, Incheon (KR); Seong Ho YUN, Incheon (KR); Seong Jin YUN, Incheon (KR)

(72) Inventors: Yeong Seok YUN, Incheon (KR); Seong Ho YUN, Incheon (KR); Seong Jin YUN, Incheon (KR)

(21) Appl. No.: 15/735,865

(22) PCT Filed: Mar. 9, 2016
(86) PCT No.: PCT/KR2016/002361

§ 371 Date: Dec. 12, 2017

(30) Foreign Application Priority Data
Aug. 17, 2015 (KR) 10-2015-0115235

(51) Int. Cl. A61B 17/34; A61B 18/08

(52) U.S. Cl. CPC A61B 17/3415; A61B 18/08; A61B 2018/00898; A61B 2017/3413; A61B 2218/002; A61B 17/3403

(57) ABSTRACT

The present invention relates to an apparatus for puncturing a maxillary sinus, the apparatus including a main body gripped by an operator and having a puncturing tube, an ultrasonic probing unit disposed in the main body and configured to detect a posterior fontanel without bones along a direction from a middle meatus to a maxillary sinus, a puncturing unit disposed in the main body, and having a needle disposed at a front end of the puncturing tube and configured to cauterize the posterior fontanel using electricity and puncture the posterior fontanel and a cautery wire configured to pass through the puncturing tube and supply electricity to the needle, an operation unit disposed in the main body and configured to move the puncturing unit toward the posterior fontanel, and an irrigation tube disposed to be fitted to the cautery wire in the puncturing tube and installed at the posterior fontanel punctured by the needle.

### TECHNICAL FIELD

The present invention relates to an apparatus for puncturing a maxillary sinus.

### BACKGROUND ART

Small cavities exist within a human's head, and the cavities are called sinuses and are connected to a nose through thin ducts, etc. Here, the sinuses include a maxillary sinus, an ethmoid sinus, a frontal sinus, a sphenoid sinus, etc.

A phenomenon in which sinuses are inflamed is called paranasal sinusitis, which is commonly referred to as sinusitis. Particularly, inflammation of a maxillary sinus is called maxillary sinusitis. Medication and surgery have been widely used to treat maxillary sinusitis.

However, when maxillary sinusitis does not respond well to medication, the maxillary sinus is punctured, an irrigation tube is injected into the maxillary sinus, the maxillary sinus is irrigated through the irrigation tube, and drugs such as antibiotics is injected into the maxillary sinus.

In this case, the use of antibiotics through oral administration or injection may be decreased, a treatment period may be shortened, maxillary sinusitis may be prevented from being aggravated to a level requiring surgery, and even patients facing difficulty using oral antibiotics due to gastrointestinal problems, etc. may be effectively treated.

Antibiotic tolerance increasing due to long-term administration of antibiotics is being recognized as a social problem. Accordingly, antibiotics locally acting only on sinuses need to be used instead of systemically-acting antibiotics, i.e. those that are orally administered or injected. Treatment using a drug delivery system, in which anticancer drugs, steroids, or antibiotics are bonded to small particles to be injected into the body, has recently gained attention. The injected drugs are gradually secreted over a month to three months and become effective. Also, the small particles are gradually decomposed and eventually disappear. The small particles are referred to as drug eluting beads. Drug eluting beads have various sizes ranging from 100 to 700 μm, and because they are formed of materials such as albumin, etc., they are safe to use in the human body and will gradually break down and disappear. When a predetermined amount of time passes after drugs are mixed with drug-eluting beads, the drugs are absorbed into the drug-eluting beads. In addition to antibiotics, drugs used for sinusitis or rhinitis may also be mixed together with drug-eluting beads. Then, an effect of taking drugs for a long period of time may be achieved by one surgical procedure, i.e. injecting the drug-eluting beads containing the drugs into the maxillary sinus. Also, even when drugs need to be taken for a long period of time, a desired effect may be achieved by acting on sinuses or nasal cavities while having almost no systemic influence, thereby significantly decreasing tolerance to drugs or possibilities of side effects.

In addition to treatment purposes, the maxillary sinus may be punctured to suction out a substance within the maxillary sinus for use in bacteriological tests or other research activities. Here, inflammation caused by anaerobic bacteria may be prevented when an irrigation tube is left within the maxillary sinus instead of being removed since oxygen is continuously supplied to the maxillary sinus through the irrigation tube.

Conventional methods for puncturing/irrigating a maxillary sinus include a method of puncturing a maxillary sinus through an inferior meatus, a method of puncturing a maxillary sinus through a canine fossa, the Proetz irrigation method and an irrigation method through a maxillary sinus ostium.

The puncturing method using the inferior meatus involves puncturing from the inferior meatus toward the maxillary sinus. A probability of failure is high when bones near a puncture are thick, a mucosal injury is a concern, and a needle for puncturing may injure eyes.

In addition, the puncturing method using the canine fossa involves puncturing toward the maxillary sinus through the oral cavity. This method has a difficulty of having to pass through the oral cavity and a concern of injuring bones at the oral mucosa and canine teeth.

In addition, in the Proetz irrigation method, a liquid medicine is injected into nasal cavities while a patient's head is leaned backward and inner portions of the nasal cavities are converted with a sound pressure to enable the liquid medicine to flow into the maxillary sinus. In this case, there is a problem of causing a discomfort for the patient.

In addition, the irrigation method through a maxillary sinus ostium is a method of injecting an irrigation tube through the maxillary sinus ostium which is an inlet from the maxillary sinus toward the middle meatus. However, there is a problem of an extreme difficulty in injecting the irrigation tube through the maxillary sinus ostium since the maxillary sinus ostium is occluded by bones, etc.

Since the conventionally known methods for puncturing/irrigating a maxillary sinus have so far failed to properly address discomfort felt by a patient and a doctor performing the surgical procedure, dangers of the surgical procedure, etc., research and development are needed.

### DISCLOSURE

### Technical Problem

The present invention has been devised to solve the above problems of the related art, and the present invention is directed to providing an apparatus for puncturing a maxillary sinus, the apparatus puncturing a posterior fontanel located in the middle meatus, thereby securing safety by not puncturing bones and facilitating the puncturing of the maxillary sinus.

In addition, the present invention is directed to providing an apparatus for puncturing a maxillary sinus, the apparatus detecting a posterior fontanel without bones using ultrasonic probing, etc., puncturing the detected posterior fontanel, and enabling an irrigation tube to be installed, thereby effectively and quickly treating maxillary sinusitis.

### Technical Solution

According to an aspect of the present invention, an apparatus for puncturing a maxillary sinus includes a main body gripped by an operator and having a puncturing tube, an ultrasonic probing unit disposed in the main body and configured to detect a posterior fontanel without bones along a direction from a middle meatus to a maxillary sinus, a puncturing unit disposed in the main body, and having a needle disposed at a front end of the puncturing tube and configured to cauterize the posterior fontanel using electricity and puncture the posterior fontanel and a cautery wire configured to pass through the puncturing tube and supply electricity to the needle, an operation unit disposed in the main body and configured to move the puncturing unit toward the posterior fontanel, and an irrigation tube disposed to be fitted to the cautery wire in the puncturing tube and installed at the posterior fontanel punctured by the needle.

Specifically, the ultrasonic probing unit may include an ultrasonic main body disposed at a rear portion of the main body, an ultrasonic probe disposed at a front portion of the main body, and a coaxial lead wire configured to transmit an electrical signal from the ultrasonic main body to the ultrasonic probe.

Specifically, the main body may further include a probing tube in a lengthy form to be inserted into the middle meatus and having the ultrasonic probe disposed at a front end thereof and the coaxial lead wire passing through an inner portion thereof, and the puncturing tube may be in a lengthy form to be inserted into the middle meatus and parallel to the probing tube.

Specifically, the operation unit may further include a mover to move the cautery wire along the puncturing tube.

Specifically, the mover may further include a moving block configured to move while fixing the cautery wire to allow the cautery wire to move along the inner portion of the puncturing tube, a rail disposed in the main body and configured to guide a movement of the moving block, and a trigger configured to move the moving block on the rail in accordance with an operator's operation.

Specifically, the mover may further include a pair of rollers configured to rotate while having the intervening cautery wire to allow the cautery wire to move along the inner portion of the puncturing tube, a rack directly or indirectly connected to the pair of rollers and configured to realize the rotation of the pair of rollers, and a trigger configured to move the rack in accordance with the operator's operation to allow the pair of rollers to rotate.

Specifically, the apparatus for puncturing a maxillary sinus may further include a rotation unit disposed in the main body and configured to allow the front end of the puncturing tube to be bent.

Specifically, the apparatus for puncturing a maxillary sinus may further include a sheath configured to hold portions of the puncturing tube besides the front end thereof being bent, and a portion of the sheath pointing toward the posterior fontanel which is a direction in which the front end of the puncturing tube is bent may be relatively thick, and other remaining portions thereof may be relatively thin.

Specifically, the sheath may include a guide structure configured to form a front end of the sheath and have a convex outer shape and a rounded inner shape, a sheath space configured to provide a place in which the front end of the puncturing tube may be bent and have a space thereof secured by the guide structure, and a sheath opening configured to provide a passage through which the needle guided by the guide structure passes.

Specifically, the apparatus for puncturing a maxillary sinus may further include an inlet portion disposed at the rear end of the puncturing tube and configured to have a cross-sectional area gradually enlarging toward the rear and form an inlet of the puncturing tube for introducing the cautery wire.

Specifically, the apparatus for puncturing a maxillary sinus may further include a notification unit configured to inform the operator of a value detected by the ultrasonic probing unit.

According to the other aspect of the present invention, a medical appliance includes the apparatus for puncturing a maxillary sinus, and a maxillary sinus tube inserted and installed at a punctured portion of the posterior fontanel punctured by the apparatus to irrigate the maxillary sinus or administer drugs into the maxillary sinus.

### Advantageous Effects

The apparatus for puncturing a maxillary sinus according to the present invention, an apparatus for puncturing a maxillary sinus punctures a posterior fontanel to install an irrigation tube in the maxillary sinus such that the maxillary sinus may be punctured without puncturing bones, thereby guaranteeing a safe surgical procedure.

In addition, the apparatus for puncturing a maxillary sinus according to the present invention, an apparatus for puncturing a maxillary sinus probes a posterior fontanel without bones using ultrasonic waves to improve convenience and a treatment time can be significantly shortened, thereby improving satisfaction of a patient and a doctor.

### BRIEF DESCRIPTION OF THE DRAWINGS

FIG. 1 is a side view of an apparatus for puncturing a maxillary sinus according to a first embodiment of the present invention;

FIG. 2 is a plan view of the apparatus for puncturing a maxillary sinus according to the first embodiment of the present invention;

FIGS. 3 and 4 are cross-sectional views taken along line A-A′ of FIG. 1;

FIG. 5 is a plan view of a puncturing tube in the apparatus for puncturing a maxillary sinus according to the first embodiment of the present invention;

FIGS. 6A and 6B are a side cross-sectional view and a plan cross-sectional view respectively illustrating another embodiment of a puncturing tube in the apparatus for puncturing a maxillary sinus according to the first embodiment of the present invention;

FIGS. 7A to 7C are sequence diagrams for describing a method of operating the puncturing tube of FIG. 6B;

FIGS. 8A and 8B are a side view and a plan view respectively illustrating another embodiment of an ultrasonic probe in the apparatus for puncturing a maxillary sinus according to the first embodiment of the present invention;

FIG. 9 is a view illustrating a cautery wire protection tube in the apparatus for puncturing a maxillary sinus according to the first embodiment of the present invention;

FIG. 10 is a view illustrating a moving block position adjuster in the apparatus for puncturing a maxillary sinus according to the first embodiment of the present invention;

FIGS. 11A to 11C are views illustrating a method of detecting a posterior fontanel by the apparatus for puncturing a maxillary sinus according to the first embodiment of the present invention;

FIGS. 12A and 12B are views illustrating a result detected in accordance with the method of detecting a posterior fontanel of FIGS. 11A to 11C;

FIG. 13 is an exploded side view of the apparatus for puncturing a maxillary sinus according to the first embodiment of the present invention;

FIGS. 14A and 14B are a side view and a plan view respectively illustrating another embodiment of an ultrasonic probing unit in the apparatus for puncturing a maxillary sinus according to the first embodiment of the present invention;

FIGS. 15A and 15B are a side view and a plan view respectively of the ultrasonic probing unit to which a probing unit cover has been added;

FIG. 16 is a view illustrating still another embodiment of an ultrasonic probing unit in the apparatus for puncturing a maxillary sinus according to the first embodiment of the present invention;

FIG. 17 is a view for describing an image shown on a monitor of the ultrasonic probing unit;

FIG. 18 is a side view of an apparatus for puncturing a maxillary sinus according to a second embodiment of the present invention;

FIG. 19 is a cross-sectional view taken along line A-A′ of FIG. 18;

FIG. 20 is a view for describing a maxillary sinus tube;

FIG. 21 is a view for describing a tube front wing of the maxillary sinus tube;

FIG. 22 is a view for describing inserting and installing the maxillary sinus tube at a punctured part of the posterior fontanel using a tube insertion tool;

FIG. 23 is a side cross-sectional view of a face; and

FIG. 24 is a front cross-sectional view of the face.

### MODE FOR THE INVENTION

The objectives, specific advantages, and new features of the present invention will become more apparent from the detailed description and the preferred embodiments below to be referenced to the accompanying drawings. In giving reference numerals to elements in each of the drawings of this specification, it should be noted that like reference numerals are given to like elements throughout the drawings. In addition, in describing the present invention, when detailed description on a known related art is deemed to unnecessarily obscure the gist of the present invention, the detailed description thereof will be omitted.

Hereinafter, the preferred embodiments of the present invention will be described in detail with reference to the accompanying drawings.

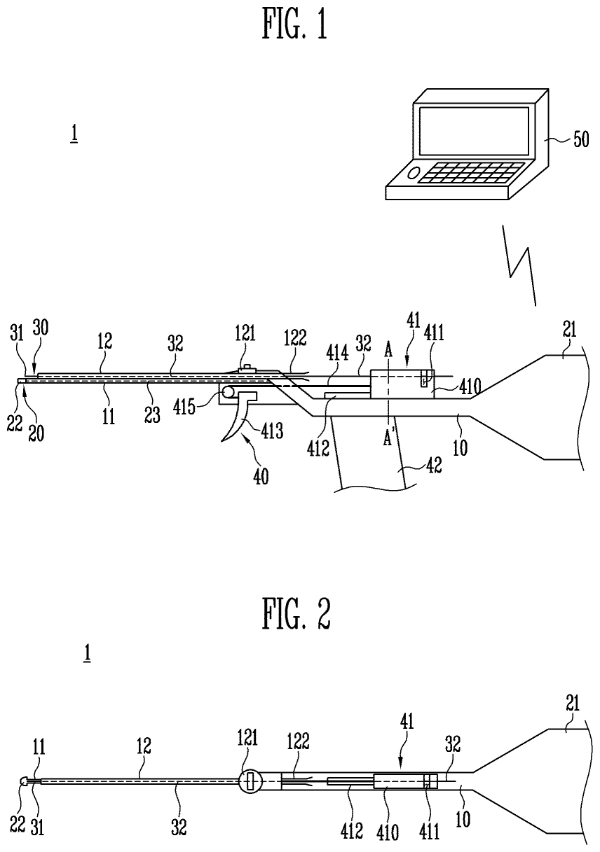

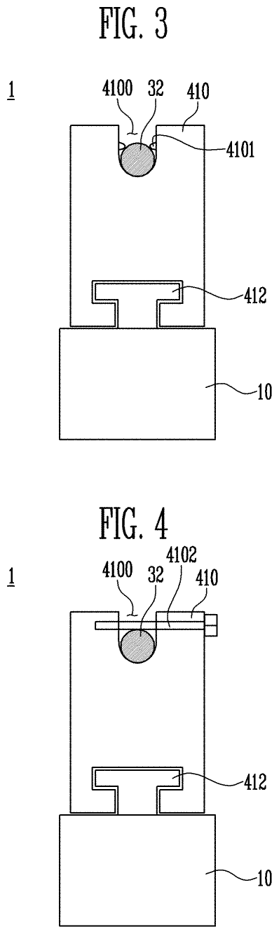

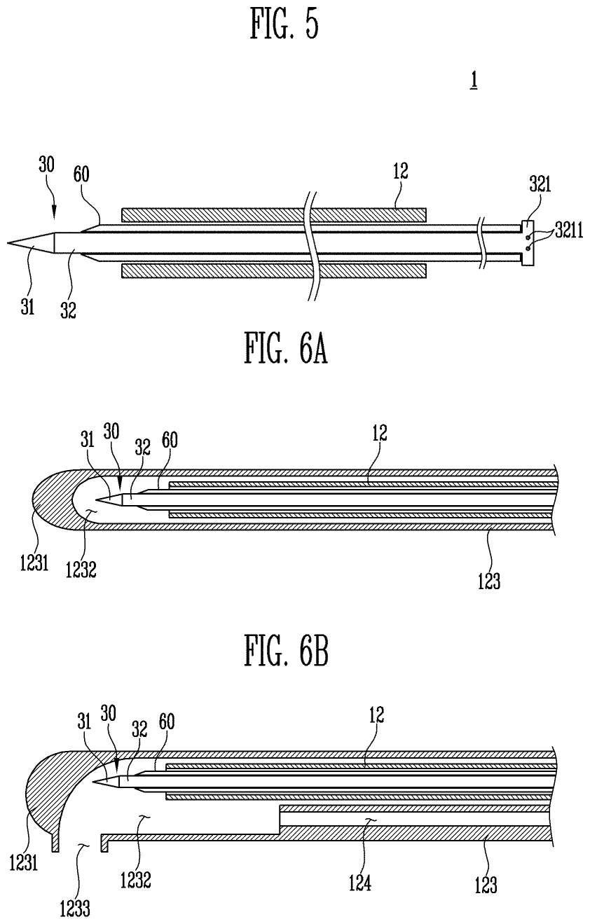

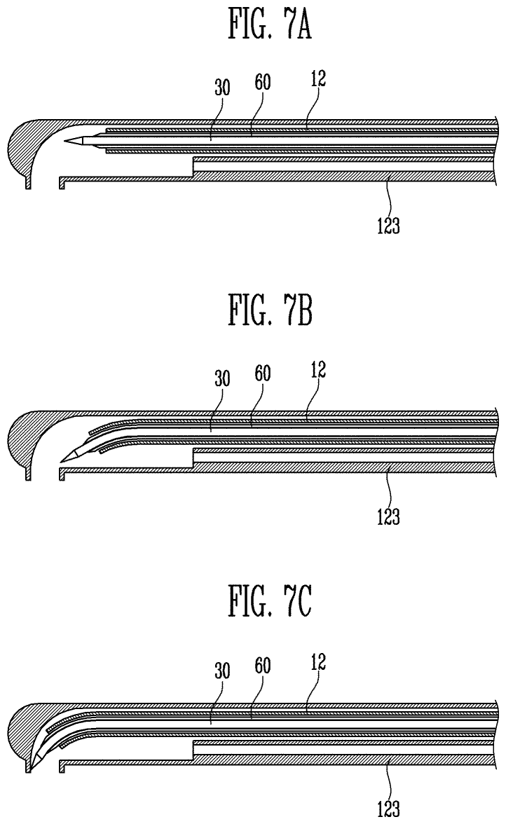

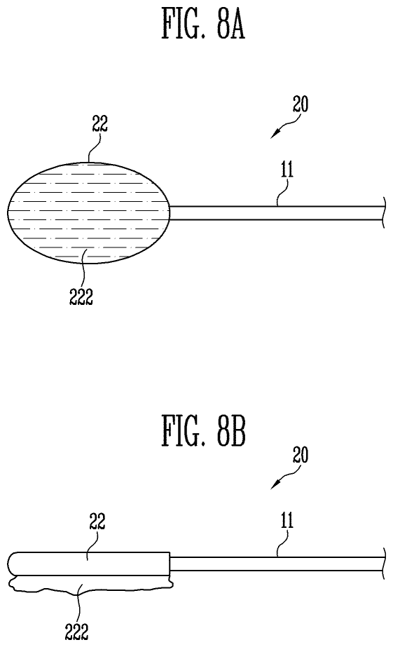

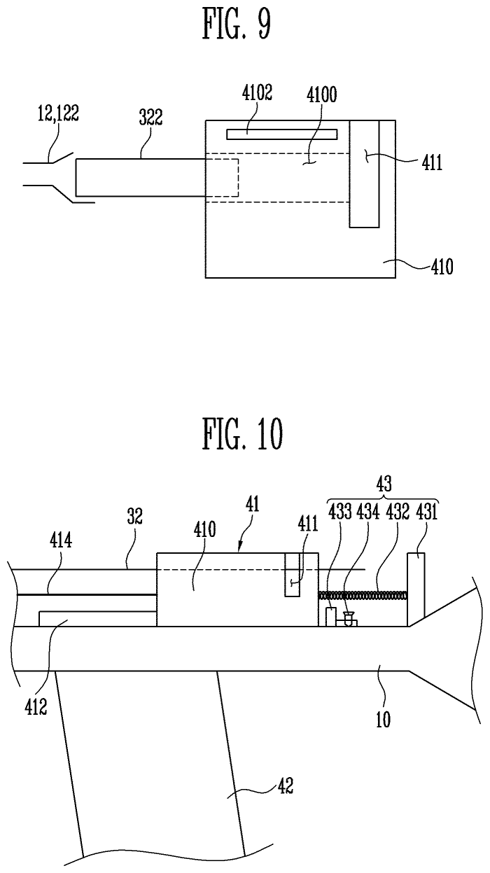

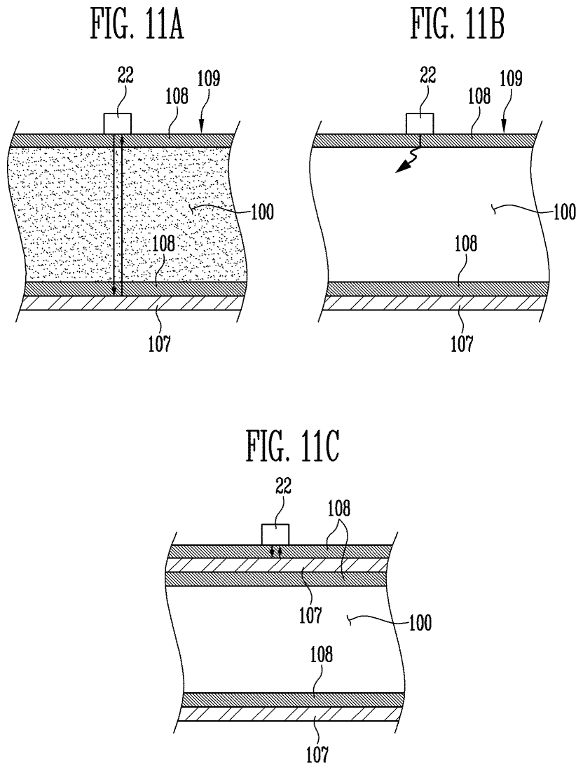

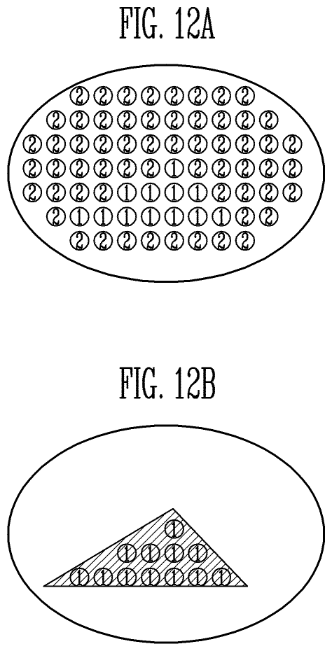

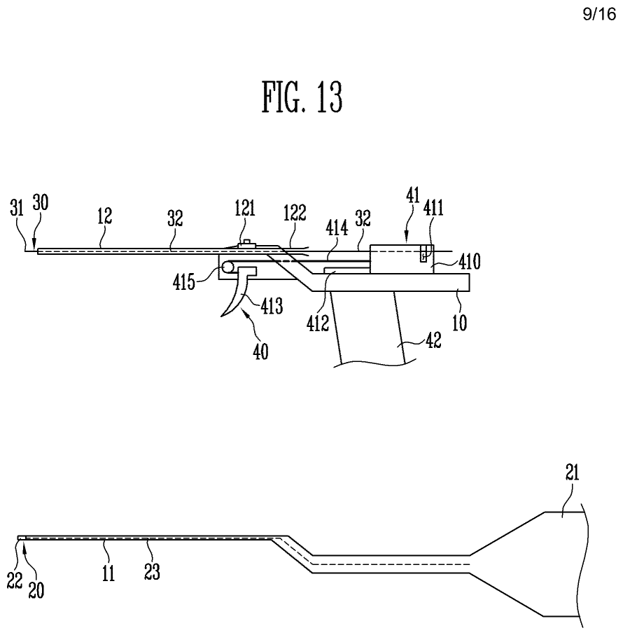

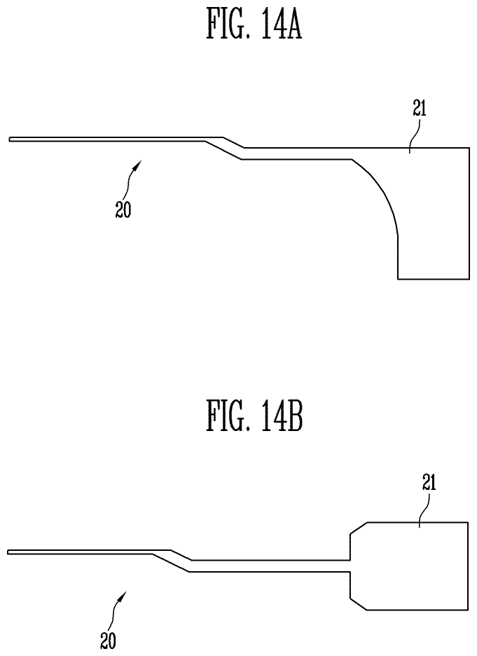

FIG. 1 is a side view of an apparatus for puncturing a maxillary sinus according to a first embodiment of the present invention, FIG. 2 is a plan view of the apparatus for puncturing a maxillary sinus according to the first embodiment of the present invention, and FIGS. 3 and 4 are cross-sectional views taken along line A-A′ of FIG. 1. In addition, FIG. 5 is a plan view of a puncturing tube in the apparatus for puncturing a maxillary sinus according to the first embodiment of the present invention, FIGS. 6A and 6B are a side cross-sectional view and a plan cross-sectional view respectively illustrating another embodiment of a puncturing tube in the apparatus for puncturing a maxillary sinus according to the first embodiment of the present invention, FIGS. 7A to 7C are sequence diagrams for describing a method of operating the puncturing tube of FIG. 6B, FIGS. 8A and 8B are a side view and a plan view respectively illustrating another embodiment of an ultrasonic probe in the apparatus for puncturing a maxillary sinus according to the first embodiment of the present invention, FIG. 9 is a view illustrating a cautery wire protection tube in the apparatus for puncturing a maxillary sinus according to the first embodiment of the present invention, FIG. 10 is a view illustrating a moving block position adjuster in the apparatus for puncturing a maxillary sinus according to the first embodiment of the present invention, FIGS. 11A to 11C are views illustrating a method of detecting a posterior fontanel by the apparatus for puncturing a maxillary sinus according to the first embodiment of the present invention, and FIGS. 12A and 12B are views illustrating a result detected in accordance with the method of detecting a posterior fontanel of FIGS. 11A to 11C.

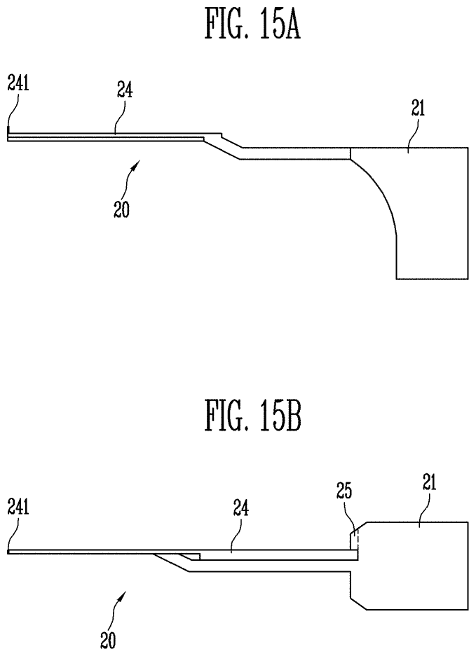

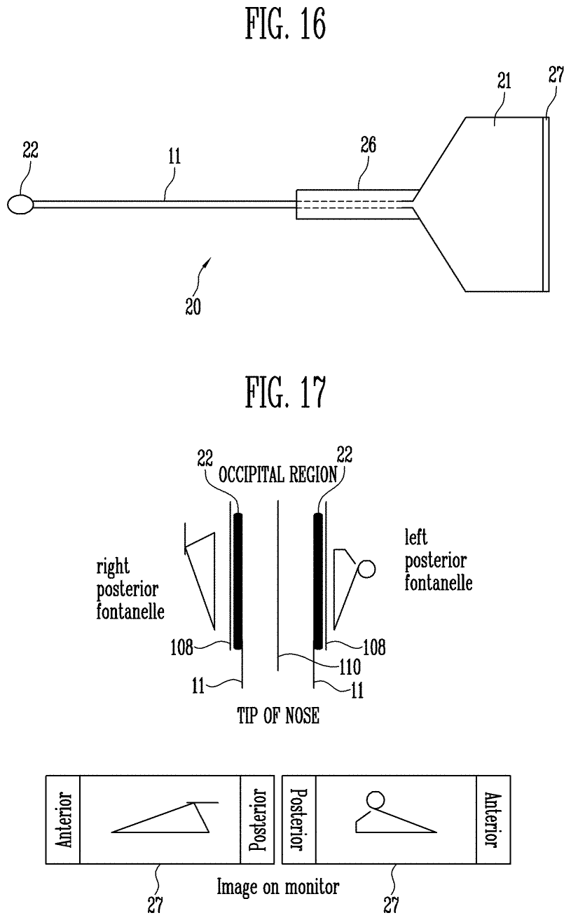

In addition, FIG. 13 is an exploded side view of the apparatus for puncturing a maxillary sinus according to the first embodiment of the present invention, FIGS. 14A and 14B are a side view and a plan view respectively illustrating another embodiment of an ultrasonic probing unit in the apparatus for puncturing a maxillary sinus according to the first embodiment of the present invention, FIGS. 15A and 15B are a side view and a plan view respectively of the ultrasonic probing unit to which a probing unit cover is added, FIG. 16 is a view illustrating still another embodiment of an ultrasonic probing unit in the apparatus for puncturing a maxillary sinus according to the first embodiment of the present invention, and FIG. 17 is a view for describing an image shown on a monitor of the ultrasonic probing unit.

In addition, FIG. 23 is a side cross-sectional view of a face, and FIG. 24 is a front cross-sectional view of the face. FIGS. 23 and 24 may be utilized as references in the description of the present invention. A maxillary sinus 100, a superior meatus 101, a superior concha 102, a middle meatus 103, a middle concha 104, an inferior meatus 105, an inferior concha 106, a posterior fontanel 109, etc. are shown in FIGS. 23 and 24.

Referring to FIGS. 1 to 17, an apparatus 1 for puncturing a maxillary sinus according to a first embodiment of the present invention includes a main body 10, an ultrasonic probing unit 20, a puncturing unit 30, an operation unit 40, a notification unit 50, and an irrigation tube 60.

The main body 10 is a part gripped by an operator. The operator may detect the posterior fontanel 109 using the ultrasonic probing unit 20 to be described below while gripping the main body 10 and then use the puncturing unit 30 to be described below to make a puncture toward the maxillary sinus 100.

The main body 10 may be in a shape of a rifle bent at least twice. This is for an index finger and a middle finger of the operator to perform different tasks while the operator is gripping the main body 10 and, at the same time, to easily secure a field of vision during a surgical procedure. Here, the operator's middle finger may be used to pull a trigger 413 of the operation unit 40 to be described below, and the index finger may be used to operate a rotation unit 121 to be described below.

In the main body 10, a direction of insertion into the middle meatus 103 through a nostril may be defined as the front, and the opposite direction may be defined as the rear. Here, a probing tube 11 and a puncturing tube 12 may be disposed at the front portion of the main body 10.

The probing tube 11 may be disposed in a lengthy form to be inserted into the middle meatus 103. The probing tube 11 is a tube disposed for ultrasonic probing, and when taking into consideration that a typical distance from a nostril to the posterior fontanel 109 is approximately 55 mm to 65 mm, the length of the probing tube 11 may be in a range of 80 mm to 100 mm. Also, the diameter of the probing tube 11 may be 1 to 3 mm.

An ultrasonic probe 22 for detecting the posterior fontanel 109 may be disposed at the front end of the probing tube 11, and a coaxial lead wire 23 for transmitting an electrical signal may be disposed inside the probing tube 11. Here, the coaxial lead wire 23 may have the front end connected to the ultrasonic probe 22 and the rear end connected to an ultrasonic main body 21 to be described below. The ultrasonic main body 21 may be disposed at a rear portion of the main body 10, and the probing tube 11 may have a structure in which the inner portion is connected to the rear portion of the main body 10.

The puncturing tube 12 may be disposed in a lengthy form to be inserted into the middle meatus 103 together with the probing tube 11 and may be vertically parallel to the probing tube 11. The puncturing tube 12 is disposed to be vertically adjacent to the probing tube 11 since nasal cavities are sufficient in the vertical direction but are limited in the horizontal direction.

The length of the puncturing tube 12 may be approximately similar to the length of the probing tube 11, and the diameter thereof may be in a range of 1.0 mm to 1.5 mm. Also, the puncturing tube 12 may be disposed to have the front end thereof rotating by the rotation unit 121. The rotation unit 121 is disposed in the main body 10 and may allow the front end of the puncturing tube 12 to be bent.

As illustrated in FIG. 5, the puncturing tube 12 may have a needle 31 disposed at the front end thereof and a cautery wire 32 for transmitting electricity to the needle 31 disposed therein. Here, the cautery wire 32 may allow the needle 31 to protrude from the front end of the puncturing tube 12 due to back and forth movements of the cautery wire 32 inside the puncturing tube 12.

When the puncturing tube 12 is inserted toward the middle meatus 103, the posterior fontanel 109 may be disposed at the left or the right of the puncturing tube 12. Accordingly, a direction of the needle 31 should be switched in order to puncture the posterior fontanel 109 using the needle 31. In the present invention, the direction of the needle 31 may be switched by enabling the front end of the puncturing tube 12 to be bent using the rotation unit 121. A principle of a fiberscope which is widely being used in a conventional endoscope may be used here. However, detailed description of the structures of the rotation unit 121 and the puncturing tube 12 using a fiberscope will be omitted.

For example, the front end of the puncturing tube 12 may be bent to the right when the rotation unit 121 is rotated clockwise, the front end of the puncturing tube 12 may be bent to the left when the rotation unit 121 is rotated counterclockwise, and a locking unit (not marked) in a bent shape to fix the front end of the puncturing tube 12 may be disposed at the rotation unit 121.

The needle 31 disposed at the front end of the puncturing tube 12 electrocauterizes the posterior fontanel 109 using electricity to puncture the posterior fontanel 109. Here, since heat is generated, the front end of the puncturing tube 12 may be insulated to be protected from the heat, and a method for insulating the front end of the puncturing tube 12 is not particularly limited.

In the above, the needle 31 punctures the posterior fontanel 109 using electricity supplied by an electrocauterizer (not illustrated), and the electrocauterizer is turned off when the needle 31 punctures the posterior fontanel 109 and enters the maxillary sinus 100. This is because pain or other side effects may be caused when the needle 31 comes in contact with mucosa 108 in the maxillary sinus 100 while the electrocauterizer is turned on.

An inlet portion 122 may be disposed at the rear end of the puncturing tube 12. The probing tube 11 allows the ultrasonic probe 22 at the front end thereof to be connected up to the ultrasonic main body 21 at the rear end thereof via the coaxial lead wire 23, and the coaxial lead wire 23 does not need to be exposed to the outside of the probing tube 11 and the main body 10 at the rear end of the probing tube 11. However, the cautery wire 32 inserted into the puncturing tube 12 may come out to the outside of the puncturing tube 12 in order to be connected to the operation unit 40 or to be replaced.

Consequently, the cautery wire 32 may be inserted into the rear end of the puncturing tube 12 toward the front end thereof through the inlet portion 122 of the puncturing tube 12. Here, the inlet portion 122 may be disposed at the rear end of the puncturing tube 12, formed in a shape of a funnel having a cross-sectional area being gradually enlarged toward the rear, and form an inlet of the puncturing tube 12 for introducing the cautery wire 32. The inlet portion 122 is formed as above in order to allow the cautery wire 323 to be easily introduced into the puncturing tube 12.

The operator may detect the posterior fontanel 109 using the ultrasonic probe 22 disposed at the front end of the probing tube 11 and then puncture the posterior fontanel 109 using the needle 31 disposed at the front end of the puncturing tube 12. However, a spot coming in contact with the ultrasonic probe 22 and a spot coming in contact with the needle 31 may be slightly different.

The puncturing tube 12 generally has a property of being slightly flexible instead of being rigid when examined by itself. As illustrated in FIGS. 6A and 6B, for preventing the puncturing tube 12 from bending when being inserted into the narrow middle meatus 103, the puncturing tube 12 may include a sheath 123 for holding portions of the puncturing tube 12 except the front end that needs to be actually bent.

The sheath 123 may be formed in a shape of a rigid tube covering the puncturing tube 12 and will be described in detail below.

The sheath 123 may be configured such that a side at which the front end of the puncturing tube 12 is bent, i.e. a portion heading toward the posterior fontanel 109, is relatively thick, and remaining portions are relatively thin. By varying the thickness of the sheath 123 as above, a space for puncturing the posterior fontanel 109 may be secured within the narrow middle meatus 103.

Since left and right sides of a patient need to be operated in order to puncture the posterior fontanel 109 with the puncturing unit 30, the sheath 123 is used while being rotated 180°. Here, to fix the sheath 123 to a position to be punctured after rotating the sheath 123, a method of forming a groove (not illustrated) on a case (not marked) surrounding a wire roller 415 and fitting a part of the sheath 123 into the groove may be used. When rotating the sheath 123, the sheath 123 may be withdrawn from the groove, rotated to a desired direction, and then fixed by being fitted into the groove again.

In addition, while holding remaining portions of the puncturing tube 12 except the front end thereof that needs to be actually bent, the sheath 123 includes a guide structure 1231, a sheath space 1232, and a sheath opening 1233 so that the front end of the puncturing tube 12 is not exposed to the outside, the front end of the puncturing tube 12 may be freely bent, and the front end of the puncturing tube 12 may accurately reach a portion of the posterior fontanel 109 to be punctured.

The guide structure 1231 may form a front end of the sheath 123 and may be configured to prevent the front end of the puncturing tube 12 from being exposed to the outside.

The guide structure 1231 may have a convex outer shape to allow the puncturing tube 12 to be easily inserted into the narrow middle meatus 103 and a rounded inner shape to allow a front end of the needle 31 to be guided along the rounded surface to be accurately positioned at the posterior fontanel 109 to be punctured.

The sheath space 1232 provides a place at which the front end of the puncturing tube 12 may be bent, and the space may be secured by the guide structure 1231.

By removing a portion of the sheath 123 at the side at which the front end of the puncturing tube 12 is bent, the sheath opening 1233 may be formed through which an end portion of the puncturing unit 30 partially exposed to the outside from the front end of the puncturing tube 12, i.e. an end portion of the needle 31 or the irrigation tube 60, is allowed to pass when the posterior fontanel 109 is punctured by the puncturing unit 30 or the posterior fontanel 109 is irrigated through the irrigation tube 60.

The sheath opening 1233 may further include an edge (not marked) protruding outside with a predetermined length at an inlet portion.

The edge forming the inlet of the sheath opening 1233 may not only serve to prevent the needle 31 or the irrigation tube 60 from being damaged due to the mucosa 108 being pushed into the sheath space 1232 when the puncturing tube 12 is rotated toward the posterior fontanel 109 after the puncturing tube 12 is inserted into the middle meatus 103, but also serve as a guide tube that allows the end portion of the needle 31 or the irrigation tube 60 that forms the end portion of the puncturing unit 30 to easily pass when the posterior fontanel 109 is being punctured or irrigated.

The sheath 123 may include an endoscope insertion passage 124 to allow an endoscope to be used when puncturing the posterior fontanel 109 by the puncturing unit 30. Here, to secure a sufficiently clear field of view when performing the surgical procedure, the sheath 123 may be manufactured with a transparent material.

The endoscope insertion passage 124 may be disposed in a shape of a tunnel in the longitudinal direction at the relatively thick portion of the sheath 123.

The endoscope insertion passage 124 may improve utilization of the apparatus 1 for puncturing a maxillary sinus.

To assist in understanding the present invention, a method of operating the puncturing tube 12 including the sheath 123 may be briefly described as follows.

Before inserting the puncturing tube 12 including the sheath 123 into nasal cavities, the front end of the puncturing tube 12 is rotated toward the thickest side of the sheath 123 and fixed, and then the puncturing tube 12 including the sheath 123 is inserted into the nasal cavities. To allow the sheath 123 to be easily inserted into the middle meatus 103 which is narrow, the sheath 123 is inserted while the narrowest width of the sheath 123 is horizontal, the widest width of the sheath 123 is vertical, and the front end of the puncturing tube 12 faces downward, and when the front end of the sheath 123 reaches a position of the posterior fontanel 109, the front end of the sheath 123 is rotated 90° such that the sheath opening 1233 turns toward the posterior fontanel 109. Since the needle 31 actually turns toward the inner portion of the maxillary sinus 100 even when the front end of the sheath 123 is slightly rotated rather than up to 90°, there is no problem in puncturing the maxillary sinus 100. Also, since the sheath 123 is rigid even when the sheath 123 receives resistance in the middle meatus 103 when the sheath 123 is being inserted, the sheath 123 is not damaged.

In other words, a space of the middle meatus 103 may be widened when the middle concha 104 is pushed toward a nasal septum, i.e. toward the center, using the sheath 123, and when the mucosa 108 in the middle meatus 103 receives a pressure and becomes thin, as much space needed for a surgical procedure may be secured as the mucosa 108 is thinned. Even though the middle meatus 103 seems to be a narrow space, when the front end of the sheath 123 is rotated 90° after the sheath 123 is inserted into the middle meatus 103 while the wide side of the sheath 123 is vertical, the rotation causing the above effects becomes possible.

Referring to FIGS. 7A to 7C, FIG. 7A illustrates an initial state of the puncturing tube 12 inserted into the sheath 123. Here, since the needle 31 of the puncturing unit 30 is extremely rigid and does not bend, the needle 31 should not be inside the puncturing tube 12 that needs to bend and should remain exposed to the outside.

FIG. 7B illustrates a state of rotating the front end of the puncturing tube 12. Here, due to a limitation on sufficiently rotating the front end of the puncturing tube 12 toward the sheath opening 1233 in the sheath space 1232, the needle 31 faces forward with a predetermined slope instead of a vertical slope. When the front end of the puncturing tube 12 is rotated and bent in a predetermined angle, the bent shape is maintained by being locked by the locking unit disposed at the rotation unit 121.

FIG. 7C illustrates a state in which the puncturing tube 12 is moved forward toward the guide structure 1231 such that the needle 31 comes in contact with an inner surface of the guide structure 1231 to be guided toward the sheath opening 1233.

FIGS. 7A to 7C described above are the process before the sheath 123 is inserted into the nasal cavities. After the process, the sheath 123 is inserted into the nasal cavities to enable the sheath opening 1233 to be located at a portion of the posterior fontanel 109 to be punctured, the sheath 123 receives the resistance of the guide structure 1231 due to continuously moving the puncturing tube 12 forward, causing the needle 31 and the cautery wire 32 to be bent further such that the needle 31 may puncture the posterior fontanel 109 by vertically passing through the sheath opening 1233.

The ultrasonic probing unit 20 is disposed in the main body 10 and detects the posterior fontanel 109 without bones 107 along a direction from the middle meatus 103 to the maxillary sinus 100. As mentioned in the discussion of the related art, a conventional method of puncturing the maxillary sinus 100 has problems in which the level of difficulty of the surgical procedure varies depending on the thickness of the bones 107, there is a concern for damaging other parts, etc., since the bones 107 are punctured. However, in the present invention, since the posterior fontanel 109 formed only with the mucosa 108 without the bones 107 are detected along the direction from the middle meatus 103 to the maxillary sinus 100 using ultrasonic waves and the posterior fontanel 109 is punctured, level of difficulty of and a danger from the surgical procedure may be significantly lowered.

Here, a value detected by the ultrasonic probing unit 20 may be shown with an image provided to the operator or indicated with a sound, a vibration, etc. The detected value may be provided to the operator in various ways by the notification unit 50 to be described below.

The ultrasonic probing unit 20 may include the ultrasonic main body 21, the ultrasonic probe 22, and the coaxial lead wire 23.

The ultrasonic main body 21 is disposed at the rear portion of the main body 10 and generates an electrical signal for generating ultrasonic waves. Since the ultrasonic main body 21 has to have the size necessary for generating and analyzing an electrical signal, the ultrasonic main body 21 may be disposed at the rear portion of the main body 10 which does not have to be inserted into the middle meatus 103. Of course the ultrasonic main body 21 may be separately provided apart from the main body 10 and connected to the main body 10 with a wire instead of being directly provided at the rear portion of the main body 10. Also, the ultrasonic main body 21 may be connected to the notification unit 50 to be described below via a wire or wirelessly and transmit a detected value to the notification unit 50.

The ultrasonic probe 22 is disposed at the front portion of the main body 10. As described above, the ultrasonic probe 22 may be disposed at the front end of the probing tube 11. Here, the ultrasonic probe 22 may detect the posterior fontanel 109 in the middle meatus 103 by converting an electrical signal into an ultrasonic wave and transmitting the ultrasonic wave.

Here, as illustrated in FIGS. 1 and 2, the ultrasonic probe 22 may be formed of a smooth, curved surface and have a form in which bent corner are minimized. This is to prevent the mucosa 108 in the nasal cavities from being damaged and to smoothly come in contact with the mucosa in the nasal cavities. The ultrasonic probe 22 may detect the posterior fontanel 109 with both corner portions.

Here, due to the ultrasonic probe 22 coming in contact also with other portions besides the portion to be detected after entering the narrow nasal cavities, a confusion in the measurement may be brought about. Consequently, a left corner and a right corner of the ultrasonic probe 22 may be made to operate independently. For example, when detecting the posterior fontanel 109 to puncture the maxillary sinus 100 at a right side with respect to a patient, probing by the right corner of the ultrasonic probe 22 may be deactivated since the left corner of the ultrasonic probe 22 may be disposed in the direction of the posterior fontanel 109, and vice versa. For this, a switch (not illustrated) for allowing only one of the right or left side of the ultrasonic probe 22 to be activated may be disposed in the main body 10 or the ultrasonic main body 21, and there may be one or more switches.

Unlike the one described with reference to FIGS. 1 and 2, the ultrasonic probe 22 according to the present invention may employ an oval disk-shaped probe as illustrated in FIGS. 8A and 8B.

The oval disk-shaped ultrasonic probe 22 may generate ultrasonic waves from one side surface, and the one side surface from which the ultrasonic waves are generated may further include a liquid structure 222.

Since the oval disk-shaped ultrasonic probe 22 may bring the one side surface having a wide area into contact with the mucosa 108 to detect the posterior fontanel 109, the detection time may be shortened.

The oval disk-shaped ultrasonic probe 22 may be a probe that applies a method of a single element transducer that converts an electrical signal into mechanical vibration by a part referred to as “piezoelectric crystal” to generate ultrasonic waves and also detects the mechanical vibration, i.e. returning ultrasonic waves, to convert the ultrasonic waves back to an electrical signal or a method of a dual element transducer that has a transmitting element and a receiving element separately provided.

Generally, since ultrasonic waves can be propagated when a probe comes in close contact with an object without an intervening air layer, the propagation of the ultrasonic waves becomes possible only when a surface of the probe is changed in accordance with the shape of the portion of the posterior fontanel 109 to come in close contact with the mucosa when attempting to use the oval disk-shaped ultrasonic probe 22 at the portion of the posterior fontanel 109 which is not flat. In this embodiment, the liquid structure 222 enables the surface of the oval disk-shaped ultrasonic probe 22 to come in close contact with the mucosa 108.

A method of using the oval disk-shaped ultrasonic probe 22 is as follows. The mucosa 108 at a portion expected to be the posterior fontanel 109 in the middle meatus 103 is marked with a needle, a central portion of the ultrasonic probe 22 is located at the marked portion of the mucosa 108, and the marked portion of the mucosa 108 is examined with ultrasonic waves to check the posterior fontanel 109 through the notification unit 50 to be described below or a monitor 27 to be described below. When the posterior fontanel 109 is actually punctured after checking a degree to which the position of the posterior fontanel 109 recognized by the ultrasonic waves is different from the portion marked with the needle, the irrigation tube 60 may be safely inserted into the maxillary sinus 100.

The coaxial lead wire 23 transmits an electrical signal from the ultrasonic main body 21 to the ultrasonic probe 22. The coaxial lead wire 23 may be disposed to pass through the inner portion of the probing tube 11 described above. That is, one end of the coaxial lead wire 23 may be connected to the ultrasonic probe 22 at the front end of the probing tube 11, and the other end of the coaxial lead wire 23 may be connected to the ultrasonic main body 21 at the rear portion of the main body 10.

The puncturing unit 30 is disposed in the main body 10 and punctures the posterior fontanel 109. Since the posterior fontanel 109 corresponds to a part formed only of the mucosa 108 and not having the bones 107, efficiency of a surgical procedure may be maximized in the present invention by penetrating only the mucosa 108 to install the irrigation tube 60 for irrigating the maxillary sinus 100.

The puncturing unit 30 may include the needle 31 and the cautery wire 32.

The needle 31 corresponds to an electrocauterizer that uses electricity to cauterize the posterior fontanel 109 and may be manufactured in a bipolar type, etc. The puncturing tube 12 may be bent by the rotation unit 121 as described above, and a direction of the needle 31 may be adjusted by the bent front end of the puncturing tube 12.

However, for the front end of the puncturing tube 12 to be easily bent by the rotation unit 121, the needle 31 formed with a rigid material may protrude from the front end of the puncturing tube 12 before the sheath 123 is inserted into a nostril or from the inside of the sheath 123.

The cautery wire 32 supplies electricity to the needle 31. The cautery wire 32 may have a structure in which an insulator is coated on a wire connected to the needle 31 and may be in a thin form to move along the inner portion of the puncturing tube 12.

The cautery wire 32 may be formed with a material that enables the cautery wire 32 to be bent together with the puncturing tube 12 being bent. However, a front end of the cautery wire 32 deviating from the front end of the puncturing tube 12 may be formed with a material capable of firmly supporting the needle 31.

That is, the material of the front end of the cautery wire 32 may simultaneously be a material capable of being bent by the rotation of the puncturing tube 12 and a material having rigidness capable of supporting the needle 31 coming out of the puncturing tube 12. Of course this may be realized by limiting materials of the cautery wire 32 and/or limiting a cross-sectional structure of the cautery wire 32, and the materials or the cross-sectional structure is not particularly limited.

A locking protrusion 321 may be disposed at the cautery wire 32. The irrigation tube 60 may be fitted to the cautery wire 32 disposed inside the puncturing tube 12, and the locking protrusion 321 of the cautery wire 32 may touch a rear end of the irrigation tube 60 in order to prevent the irrigation tube 60 from moving backward when the cautery wire 32 moves forward. Here, the locking protrusion 321 may be disposed to be positioned within an open groove 411 to be described below.

A cauterizing main body (not illustrated) for transmitting electricity may be connected to a rear end of the cautery wire 32, and like the ultrasonic main body 21 described above, the cauterizing main body may be disposed in the main body 10 or disposed to be spaced apart from the main body 10 and connected to the main body 10 via a wire.

The cautery wire 32 may be manufactured to reach only up to the locking protrusion 321 and be fixed, and here, as illustrated in FIG. 5, a socket 3211 connected to the cautery wire 32 may be disposed at the locking protrusion 321. The socket 3211 may be connected to or disconnected from an external wire and may be manufactured in various shapes at a portion of the locking protrusion 321. However, when a spring 432 to be described below is disposed at a rear portion of a moving block 410 to be described below, the socket 3211 is preferably disposed at an upper end of the locking protrusion 321 as illustrated in FIG. 5 to avoid interfering with the spring 432.

In addition, a portion of the cautery wire 32 extending from the inlet portion 122 of the puncturing tube 12 up to the moving block 410 to be described below may be exposed to the outside instead of being protected by the puncturing tube 12, and the exposed cautery wire 32 may be bent when the moving block 410 moves forward. Accordingly, as illustrated in FIG. 9, a cautery wire protection tube 322 may be additionally disposed between the inlet portion 122 of the puncturing tube 12 and the moving block 410 to be described below in order to protect the exposed cautery wire 32.

The cautery wire protection tube 322 may have one end portion inserted into a fixing groove 4100 of the moving block 410 to be described below and be fixed to extend up to the inlet portion 122 of the puncturing tube 12 and may prevent the cautery wire 32 from being bent when the moving block 410 moves forward.

The operation unit 40 is disposed in the main body 10 and moves the puncturing unit 30 toward the posterior fontanel 109. The operation unit 40 is a configuration for allowing the needle 31 to come in contact with the posterior fontanel 109 to puncture the posterior fontanel 109, and specifically, the operation unit 40 may allow the cautery wire 32 to move forward in the puncturing tube 12.

The operation unit 40 may include a mover 41, a main body handle 42, and a moving block position adjuster 43.

The mover 41 may move the cautery wire 32 along the puncturing tube 12, and particularly, the mover 41 may move the cautery wire 32 forward along the puncturing tube 12. When the cautery wire 32 moves forward, the needle 31 connected to the front end of the cautery wire 32 may touch the posterior fontanel 109.

The mover 41 may include the moving block 410, a rail 412, and the trigger 413.

The moving block 410 moves the cautery wire 32 which is fixed to allow the cautery wire 32 to move along the inner portion of the puncturing tube 12. The moving block 410 may include the fixing groove 4100 recessed from an upper surface thereof in the longitudinal direction of the puncturing tube 12, and when the cautery wire 32 is fitted into the fixing groove 4100, a movement of the moving block 410 may be entirely transmitted to the cautery wire 32.

Here, to allow the cautery wire 32 to be fixed in a state of being fitted into the fixing groove 4100 of the moving block 410, a fixing protrusion 4101 may be disposed at the fixing groove 4100 as illustrated in FIG. 3. The fixing protrusion 4101 may prevent the cautery wire 32 from being detached from the fixing groove 4100 when the cautery wire 32 is completely inserted into the fixing groove 4100, and although, in this case, the fixing protrusion 4101 may of course serve as an obstacle in the process of inserting the cautery wire 32 into the fixing groove 4100, the cautery wire 32 may be completely inserted into the fixing groove 4100 over the fixing protrusion 4101 when the cautery wire 32 is pressed by applying a predetermined force.

A fixing member 4102 may be fitted into the fixing groove 4100 as illustrated in FIG. 4. That is, when the fixing member 4102 is fitted to the fixing groove 4100 by the operator to cross an upper portion of the cautery wire 32 after the cautery wire 32 or the cautery wire protection tube 322 is inserted into the fixing groove 4100, the cautery wire 32 or the cautery wire protection tube 322 may be prevented from being detached upward. Here, the fixing member 4102 may have a pin type structure or a plate type structure, one or more of the fixing member 4102 may be disposed along the longitudinal direction of the cautery wire 32, and a groove (not marked) for inserting the fixing member 4102 may be disposed at the moving block 410.

When the fixing member 4102 has a pin type structure, the fixing member 4102 may have a screw form having a screw thread or a simple pin form without a screw thread, and the groove may be disposed in a shape corresponding to the form of the fixing member 4102. When the fixing member 4102 is rotated by a predetermined angle, the fixing member 4102 is changed to being removable from the groove or not being removable, thereby separating or fixing of the fixing member 4102 may be accomplished. This may be realized by a structure in which a protrusion (not illustrated) is disposed at a predetermined position outward from the fixing member 4102 and the fixing member 4102 may be inserted/withdrawn only when the corresponding protrusion is aligned to a specific position.

The open groove 411 may be disposed at the moving block 410. The open groove 411 is in a form of a groove that is recessed deeper than the fixing groove 4100 and may be opened sideward to allow the cautery wire 32 to be exposed from a side surface. Consequently, the operator may use the open groove 411 to check from the side surface whether the cautery wire 32 is properly fixed to the moving block 410. The open groove 411 may be disposed at a middle point of the fixing groove 4100 disposed from the front end to the rear end of the moving block 410.

In addition, the locking protrusion 321 of the cautery wire 32 may be fitted to the open groove 411. Since the cautery wire 32 at which the locking protrusion 321 is provided moves forward by the moving block 410 at which the open groove 411 is disposed, the open groove 411 may have a horizontal width that allows the locking protrusion 321 to be fitted thereto. Here, the irrigation tube 60 may have a length in which the rear end thereof extends up to the open groove 411.

The rail 412 is disposed in the main body 10 and guides a movement of the moving block 410. The rail 412 may have a T-shaped cross-section, and a rail groove (not marked) into which the rail 412 is inserted may be disposed at the moving block 410 such that the moving block 410 may move back and forth while remaining fitted to and not detaching from the rail 412.

Here, a roller or a sliding surface that facilitates a movement of the moving block 410 may be disposed at the rail 412, and the moving block 410 may remain spaced apart from an upper surface of the main body 10 while moving along an upper surface of the rail 412 to prevent the movement of the moving block 410 from being limited due to friction.

The rail 412 may have a form that horizontally extends by as much distance as is needed for the cautery wire 32 to move for the needle 31 to touch the posterior fontanel 109. Here, a step (not illustrated) to prevent the moving block 410 from being detached may be disposed on at least one of a front end and a rear end of the rail 412. Of course the step may be disposed on the upper surface of the rail 412 and/or the upper surface of the main body 10.

The trigger 413 moves the moving block 410 on the rail 412 in accordance with the operator's manipulation. The trigger 413 may be connected to the moving block 410 via a wire 414, and the wire 414 may be wound around the wire roller 415 disposed in front of the trigger 413. When the trigger 413 is moved backward by the operator's middle finger, the wire roller 415 is rotated and the wire 414 is pulled such that the moving block 410 may move forward.

Of course a structure and a principle of the cautery wire 32 moving forward by the action of pulling the trigger 413 is not limited to the above in the present invention. In a second embodiment below, the cautery wire 32 may be moved forward by a roller 416 instead of the moving block 410 when the trigger 413 is pulled. This will be described below.

The main body handle 42 may be disposed at a portion of the main body 10 in a form of a handle of a pistol for the operator to easily grip the main body 10.

The moving block position adjuster 43 may be installed at a rear portion of the moving block 410 as illustrated in FIG. 10 to adjust a position of the moving block 410 and includes a fixed wall 431, the spring 432, a preventive wall 433, and a screw 434.

The spring 432 may be installed between the rear end of the moving block 410 and the fixed wall 431 installed by being spaced apart from the moving block 410 by a predetermined distance.

The length of the cautery wire 32 may be slightly increased or decreased while the apparatus 1 for puncturing a maxillary sinus is being used. The moving block position adjuster 43 is provided for compensating for the increased or decreased length and prevents the moving block 410 from moving from a predetermined position.

To manage the predetermined position of the moving block 410 more quantitatively, the preventive wall 433 that prevents the moving block 410 from moving further even when the spring 432 pulls the moving block 410 from the rear of the moving block 410 is provided. The preventive wall 433 is fixed by the screw 434. A few preventive walls with different thicknesses may be prepared for the preventive wall 433, and when the moving block 410 needs to be placed more forward or backward due to a changed length of the cautery wire 32, the preventive wall 433 may be replaced and a preventive wall corresponding to the changed length may be installed.

The notification unit 50 informs the operator of a value detected by the ultrasonic probing unit 20. The notification unit 50 may include a display for showing the value detected by the ultrasonic probe 22 with an image.

In addition, the notification unit 50 may inform the operator of the detected value in a simpler way, and this will be described with reference to FIGS. 11A to 11C and FIGS. 12A and 12B.

Referring to FIGS. 11A to 11C, the ultrasonic probe 22 may transmit ultrasonic waves and receive the returning ultrasonic waves. Here, when a duration of time from a time of transmitting the ultrasonic waves up to a time of receiving the ultrasonic waves is measured, a distance of a portion to which the ultrasonic waves may be propagated may be figured out.

However, the ultrasonic waves have a property of being unable to pass through the bones 107 despite being able to pass through the mucosa 108 and has a property of being unable to be propagated when only air is present in the maxillary sinus 100 despite being able to propagate when a liquid such as a secretion is filled within the maxillary sinus 100.

FIG. 11A is a case in which ultrasonic waves are propagated from the posterior fontanel 109 formed only of the mucosa 108 without the bones 107 in the middle meatus 103 by the ultrasonic probe 22 while a liquid such as secretions is filled within the maxillary sinus 100. The ultrasonic waves return by the bones 107 inside the maxillary sinus 100 after passing through the maxillary sinus 100, and here, the time consumed by the ultrasonic waves to return may be expressed as a long time “Ta.”

FIG. 11B is a case in which ultrasonic waves are propagated from the posterior fontanel 109 formed only of the mucosa 108 without the bones 107 in the middle meatus 103 by the ultrasonic probe 22 while only air is present in the maxillary sinus 100. The ultrasonic waves disappear within the maxillary sinus 100, and here, the time consumed by the ultrasonic waves to return may be expressed as an infinite time “Tb.”

FIG. 11C is a case in which ultrasonic waves are propagated from portions where the bones 107 are present in the middle meatus 103 by the ultrasonic probe 22. The ultrasonic waves immediately return without passing through the maxillary sinus 100, and here, the time consumed by the ultrasonic waves to return may be expressed as a short time “Tc.”

That is, like Ta, Tb, and Tc, differences may occur in time taken for recovering ultrasonic waves depending on whether the bones 107 are present in the spot from which the ultrasonic waves are propagated and an inner state of the maxillary sinus 100, and the ultrasonic probing unit 20 may detect the posterior fontanel 109 using this principle. In this case, the notification unit 50 does not have to show the detected value with an image, and may instead confirm the user of whether the bones 107 are present and whether a liquid or air is filled within the maxillary sinus 100 using a simple value that shows a distance reached by the ultrasonic waves or using different sounds/vibrations with respect to FIG. 11A, FIG. 11B, and FIG. 11C. Here, since the notification unit 50 does not require the display, a size of equipment of the present invention may be reduced and the notification unit 50 may be disposed within the ultrasonic main body 21.

That is, the operator may recognize a position of the posterior fontanel 109 from the notification unit 50 while detecting the posterior fontanel 109 by the ultrasonic probing unit 20 and then puncture the posterior fontanel 109, thereby conveniently performing the surgical procedure.

As illustrated in FIGS. 12A and 12B, a result detected in accordance with the method of detecting the posterior fontanel described with reference to FIGS. 11A to 11C may be shown with an image on a monitor. As illustrated in FIG. 12A, a number “1” is displayed on the monitor when the time of recovering ultrasonic waves is either the long time “Ta” or the endless time “Tb,” and a number “2” is displayed on the monitor when the time of recovering the ultrasonic waves is the short time “Tc.” When the long times “Ta” and the endless times “Tb” are grouped by the number “1” and the short times “Tc” are grouped by the number “2” as above and colors of the numbers are different, a form of the posterior fontanel 109 may be shown with a color image on the monitor. In addition, as illustrated in FIG. 12B, the number “2” may be erased and only the number “1” may remain to show the form of the posterior fontanel 109 with an image on the monitor with a shape thereof.

The irrigation tube 60 may be disposed to be fitted to the cautery wire 32 in the puncturing tube 12 and may be installed at the posterior fontanel 109 punctured by the needle 31. The irrigation tube 60 is a configuration for connecting the inside and the outside of the maxillary sinus 100, and after the posterior fontanel 109 is punctured by the puncturing unit 30, the irrigation tube 60 may remain installed through the punctured hole. The irrigation tube 60 is installed for irrigating the maxillary sinus 100 and may be used to administer drugs as needed.

The irrigation tube 60 may be manufactured using a material that is bendable while being rigid to a certain degree and harmless to the human body, and there is no problem in irrigating the maxillary sinus 100 even when the diameter of the irrigation tube 60 is smaller than 1 mm. A front end portion of the irrigation tube 60 is tapered similarly to a tube catheter used when performing an intravenous injection such that resistance may be minimized when the irrigation tube 60 passes through the hole together with the needle 31.

The irrigation tube 60 is installed at the punctured hole of the posterior fontanel 109. The needle 31 may move forward for puncturing the posterior fontanel 109 and move forward for installing the irrigation tube 60 after puncturing the posterior fontanel 109. The needle 31 in the former case moves forward only until it touches the posterior fontanel 109, but the needle 31 in the latter case may move forward until it enters the maxillary sinus 100.

In the latter case, when the needle 31 and the irrigation tube 60 enter the maxillary sinus 100 through the hole of the posterior fontanel 109, the operator may move only the needle 31 and the cautery wire 32 backward. Here, the irrigation tube 60 may come out forward from the cautery wire 32 and be fixed to the hole.

Since the hole of the posterior fontanel 109 formed by the needle 31 may naturally be blocked as time passes after the irrigation tube 60 is removed, thus is not a problem. Also, even when the hole is not blocked, it is not a problem since there are cases in which an accessory ostium is present in a normal person.

The structure of the apparatus 1 for puncturing a maxillary sinus according to the present invention described above may be referred to as an integral structure. However, the apparatus 1 for puncturing a maxillary sinus according to the present invention may also be configured in a detachable structure in which the ultrasonic probing unit 20 may be detached from the main body 10 in order to puncture the posterior fontanel 109 in the narrow nasal cavities or irrigate the maxillary sinus 100 or facilitate drug administration through the punctured portion and to easily detect the posterior fontanel 109. The apparatus 1 for puncturing a maxillary sinus manufactured in the detachable structure will be described with reference to FIG. 13.

As illustrated in FIG. 13, the apparatus 1 for puncturing a maxillary sinus manufactured in the detachable structure may be configured with the main body 10 including the puncturing unit 30 and the operation unit 40, and the ultrasonic probing unit 20 detached from the main body 10, and may separately perform detection or a puncturing procedure. The apparatus 1 for puncturing a maxillary sinus manufactured in the detachable structure may of course be combined with the ultrasonic probing unit 20 to perform the detection or the puncturing procedure.

When the apparatus 1 for puncturing a maxillary sinus is manufactured in the integral structure, the apparatus 1 for puncturing a maxillary sinus needs to be configured to be able to rotate the sheath 123 surrounding the puncturing tube 12 in order to perform ultrasonic probing or puncturing with respect to left and right sides of a patient, and the ultrasonic probing unit 20 needs to be configured in one left and right pair when the ultrasonic probe 22 is in the oval disk shape in which ultrasonic waves are generated from one side surface as illustrated in FIG. 8. However, the apparatus 1 for puncturing a maxillary sinus manufactured in the detachable structure may be configured such that the sheath 123 surrounding the puncturing tube 12 does not have to be rotated in order to perform ultrasonic probing or puncturing with respect to left and right sides of the patient and only one ultrasonic probing unit 20 is disposed instead of the one left and right pair thereof regardless of the type of the ultrasonic probe 22.

In addition, while the front end of the puncturing tube 12 is configured to be bent 90° sideward with respect to the direction of the trigger 413 in the apparatus 1 for puncturing a maxillary sinus manufactured in the integral model, the front end of the puncturing tube 12 may be configured to be bent toward the trigger 413 in the apparatus 1 for puncturing a maxillary sinus manufactured in the detachable model.

In the apparatus 1 for puncturing a maxillary sinus manufactured in the detachable model, the puncturing unit 30 is inserted into the middle meatus 103 while the side at which the width of the sheath 123 is wide is vertical, i.e. while the trigger 413 and the main body handle 42 are vertical, when inserting the puncturing tube 12 into the middle meatus 103, and the main body handle 42 is rotated when puncturing the posterior fontanel 109 such that the sheath opening 1233 of the sheath 123 and the front end of the puncturing tube 12 turns toward the posterior fontanel 109. Here, the front end of the puncturing tube 12 turns toward the posterior fontanel 109 at the right side of the patient when the main body handle 42 is rotated 90° clockwise, and the front end of the puncturing tube 12 turns toward the posterior fontanel 109 at the left side of the patient when the main body handle 42 is rotated 90° counterclockwise such that the posterior fontanel 109 at the left side or the right side may be punctured.

In the apparatus 1 for puncturing a maxillary sinus manufactured in the detachable model, although the ultrasonic probing unit 20 does not have the main body handle 42 that may be seen in the apparatus 1 for puncturing a maxillary sinus manufactured in the integral model, the ultrasonic probing unit 20 has the main body handle 42 formed in a shape similar to the main body 10 as a whole to be gripped as if holding a pencil and rotated to detect the posterior fontanel 109 at the left side or the right side.

The detachable ultrasonic probing unit 20 in the apparatus 1 manufactured in the detachable model may include a groove (not illustrated) into which the rear end of the main body 10 may be fitted and fixed formed at a front surface portion of the ultrasonic main body 21 in order to facilitate the detachment of the ultrasonic probing unit 20 from the main body 10. Also, when being attached to the main body 10, the ultrasonic probing unit 20 may be configured such that a rear portion of the probing tube 11 may be surrounded by a clip (not illustrated) and coupled to the puncturing tube 12 to prevent the probing tube 11 from being detached from the puncturing tube 12.

In addition, as illustrated in (a) and (b) of FIG. 15, the ultrasonic probing unit 20 in the apparatus 1 manufactured in the detachable model may include a probing unit cover 24 for the ultrasonic probing unit 20 to be easily gripped and protected. The probing unit cover 24 extends up to an upper portion of the probing tube 11, and this portion is preferably manufactured in a plate shape to reduce the volume.

A marking needle 241 may be disposed at one end portion of the probing unit cover 24, i.e. a portion at which the ultrasonic probe 22 is disposed, in order to mark a portion of the posterior fontanel 109 to be punctured. When the posterior fontanel 109 is checked by the ultrasonic probe 22 and the ultrasonic probing unit 20 is rotated 90°, the marking needle 241 leaves a scratch on the mucosa 108 of the posterior fontanel 109 to mark a portion to be punctured.

To facilitate a detachment of the probing unit cover 24, a coupling groove 25 may be disposed at the front surface portion of the ultrasonic main body 21 to allow a rear end of the probing unit cover 24 to be fitted thereto and fixed. Also, a rear portion of the probing unit cover 24 may be surrounded by a clip (not illustrated) and coupled to the probing tube 11 to prevent the attached probing unit cover 24 from being detached from the probing tube 11.

The ultrasonic main body 21 may be manufactured in various shapes in the apparatus 1 for puncturing a maxillary sinus manufactured in the integral model or the apparatus 1 for puncturing a maxillary sinus manufactured in the detachable model. However, when the socket 3211 is disposed at the locking protrusion 321 as illustrated in FIG. 5, the upper end portion of the ultrasonic main body 21 may be manufactured in a flat shape to be coplanar with the upper end portion of the main body 10 as illustrated in FIGS. 14A and 14B in order to remove an inconvenience when connecting an external wire to the socket 3211.

The ultrasonic probing unit 20 in the apparatus 1 manufactured in the detachable model may be configured to further include a probing unit handle 26 at a front portion of the ultrasonic main body 21 and a monitor 27 at a rear portion of the ultrasonic main body 21 as illustrated in FIG. 16.

When the probing unit handle 26 is gripped as if holding a pencil, the ultrasonic probing unit 20 in the apparatus 1 manufactured in the detachable model may allow performing reliable ultrasonography. Here, when a surface of the probing unit handle 26 is angularly formed, the ultrasonic probing unit 20 may be prevented from freely rotating when gripped.

The ultrasonic probe 22 may be a probe in which ultrasonic waves are generated from one surface instead of being generated from left and right sides, e.g., an oval disk-shaped probe in which ultrasonic waves are generated from one side surface as illustrated in FIG. 8. To check the left side or the right side of the patient, the probing unit handle 26 just has to be held while rotating the ultrasonic probing unit 20 by 180° without other manipulations. The ultrasonography may begin and end by a button (not illustrated) or by a voice recognition method since the operator performs nasoendoscopy with one hand and uses the ultrasonic probing unit 20 with the other hand.

When the probing unit handle 26 is held while rotating the ultrasonic probing unit 20 by 180° with respect to an axis of the probing unit handle 26, the ultrasonic probe 22 also turns toward the opposite direction, and the monitor 27 disposed at the rear portion of the ultrasonic main body 21 also rotates 180° at the same time. Then, the posterior fontanel 109 may be naturally viewed from the monitor 27 in a form necessary when performing the surgical procedure without other manipulations as illustrated in FIG. 17. Generally, for an anatomical depiction, left and right structures are depicted as seen from the nasal septum 110, i.e. the center of the nasal cavities, and since images shown on the monitor 27 are the same as above, there is no inconvenience in performing the surgical procedure. One pair of monitors 27 are seen in FIG. 17. One of the monitors 27 shows a state of rotating the other monitor 27 by 180°. Here, “anterior” refers to a direction toward the tip of a nose, and “posterior” refers to a direction toward an occipital region.

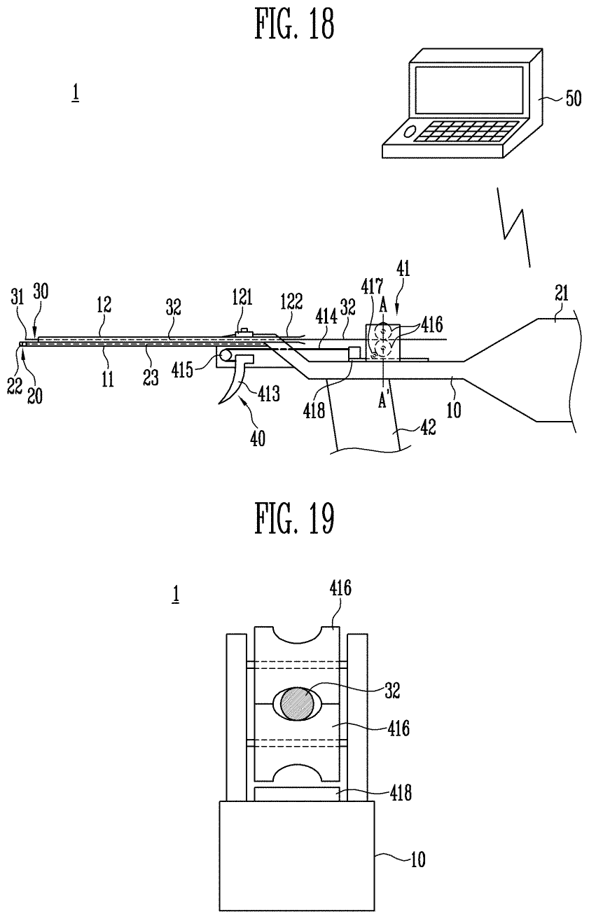

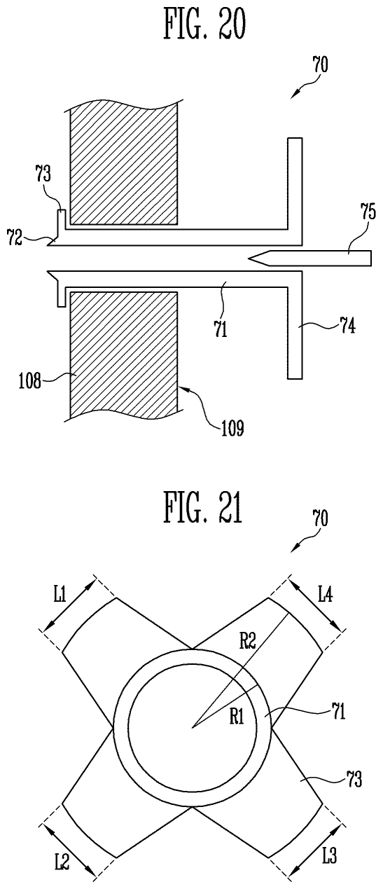

Hereinafter, another embodiment in which the mover 41 becomes different will be described with reference to FIGS. 18 and 19.

FIG. 18 is a side view of an apparatus for puncturing a maxillary sinus according to the second embodiment of the present invention, and FIG. 19 is a cross-sectional view taken along line A-A′ of FIG. 18.

Referring to FIGS. 18 and 19, the mover 41 of the apparatus 1 for puncturing a maxillary sinus according to the second embodiment of the present invention may include the roller 416, a rack 418, and the trigger 413. Hereinafter, differences from the first embodiment will be mainly described, and description of the parts overlapping with the first embodiment will be omitted.

The roller 416 may be configured in one pair. The one pair of rollers 416 may rotate while having an intervening cautery wire 32 to allow the cautery wire 32 to move along the inner portion of the puncturing tube 12. As illustrated in the drawings, the rollers 416 may be vertically disposed with respect to the cautery wire 32 or horizontally disposed with respect to the cautery wire 32.

Portions of the rollers 416 touching the cautery wire 32 may be recessed to prevent the cautery wire 32 from being detached to the outside, and the two rollers 416 may touch each other while having an intervening cautery wire 32. In this case, when the two rollers 416 touch each other, the size of the recessed portions may correspond to the cross-sectional area of the cautery wire 32. Also, the rollers 416 may be formed with an elastic material to touch both of the cautery wire 32 and the other roller 416.

At least one of the pair of rollers 416 may be rotated by the trigger 413. Since the pair of rollers 416 may touch each other, with only one of the rollers 416 rotated by the trigger 413, both of the rollers 416 may rotate and push the cautery wire 32 forward.

The rack 418 is directly or indirectly connected to the rollers 416 to realize the rotations of the rollers 416. The rack 418 may be disposed on the upper surface of the main body 10 and disposed to move in the horizontal direction. The movement of the rack 418 may be realized by the same/similar principle and structure with the movement of the moving block 410 mentioned above.

The rack 418 may move forward when the trigger 413 is pulled, and when the rack 418 moves forward, the rollers 416 may rotate and move the cautery wire 32 forward. Here, to align a direction in which the rack 418 moves and a direction in which the rollers 416 rotate, an auxiliary roller 417 may be connected between the rack 418 and the rollers 416. That is, the rack 418 may transmit a rotary force to the auxiliary roller 417, and the rollers 416 may rotate by touching the auxiliary roller 417. Consequently, when the rack 418 moves forward, the auxiliary roller 417 may move clockwise, a lower roller 416 of the pair of rollers 416 may move counterclockwise, and an upper roller 416 may move clockwise by touching the lower roller 416. Thus, the cautery wire 32 between the pair of rollers 416 moves forward.

The trigger 413 moves the rack 418 in accordance with the operator's manipulation to rotate the rollers 416. Since the wire 414 and the wire roller 415, etc. described in the first embodiment above may be wholly applied to the rack 418 moving by the trigger 413, the detailed description thereof will be omitted.

In this embodiment, the rollers 416, the auxiliary roller 417, etc. are configurations rotating by friction force. However, different from above, the rollers 416 and the auxiliary roller 417 may be substituted with gears rotating by interlocking gear teeth in the present invention. In this case, gear teeth may be disposed at one surface of the rack 418, and the auxiliary roller 417 may be a rack gear interlocking with the rack 418.

Hereinafter, an order of a surgical procedure using the apparatus 1 for puncturing a maxillary sinus manufactured in the integral structure will be briefly described.

While gripping the main body 10, the operator may check that the ultrasonic probe 22 and the coaxial lead wire 23 are disposed at the probing tube 11 and the needle 31 and the cautery wire 32 are disposed at the puncturing tube 12.

Then, the operator may simultaneously insert the probing tube 11 and the puncturing tube 12 into the middle meatus 103 and detect the posterior fontanel 109 in the middle meatus 103 using the ultrasonic probe 22 to locate a portion without the bones 107.

Then, to puncture the posterior fontanel 109, the operator may adjust a direction of the front end of the puncturing tube 12 and pull the trigger 413. When the trigger 413 is pulled, the needle 31 and the cautery wire 32 move forward such that the needle 31 is located at a desired portion and a hole is formed by electrocautery.

Then, to insert the irrigation tube 60 through the hole, the operator may pull the trigger 413 again to allow the needle 31 and the irrigation tube 60 to be inserted into the maxillary sinus 100 through the hole. Here, the trigger 413 may be pulled further than when the trigger 413 is pulled for puncturing the posterior fontanel 109. The trigger 413 which is pulled when puncturing the posterior fontanel 109 may return to an original position by a spring (not illustrated) disposed at the trigger 413, and then the trigger 413 may be pulled for inserting the irrigation tube 60.

Then, to allow the irrigation tube 60 disposed in the hole to remain inside the maxillary sinus 100, the operator may carefully take out only the needle 31. By this process, the irrigation tube 60 communicating with the outside may be installed in the maxillary sinus 100.

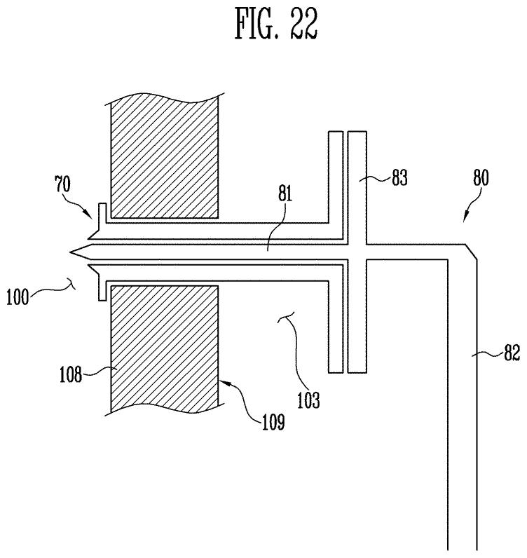

The apparatus 1 for puncturing a maxillary sinus according to the first embodiment or the second embodiment may detect the posterior fontanel 109 by the ultrasonic probing unit 20, puncture the posterior fontanel 109 by the puncturing unit 30, and irrigate the maxillary sinus 100 or administer drugs by the irrigation tube 60. However, when irrigating or administering drugs by the irrigation tube 60 once is deemed to have only a marginal effect, the irrigation or the drug administration should be performed several times. Each time, continuously irrigating the maxillary sinus 100 or administering drugs to the maxillary sinus 100 through the hole formed at the posterior fontanel 109 after the irrigation tube 60 is removed may be more preferable than continuing to puncture the posterior fontanel 109. In the present invention, parts of the apparatus 1 for puncturing a maxillary sinus to solve the above problem will be described with reference to FIGS. 20 to 22.

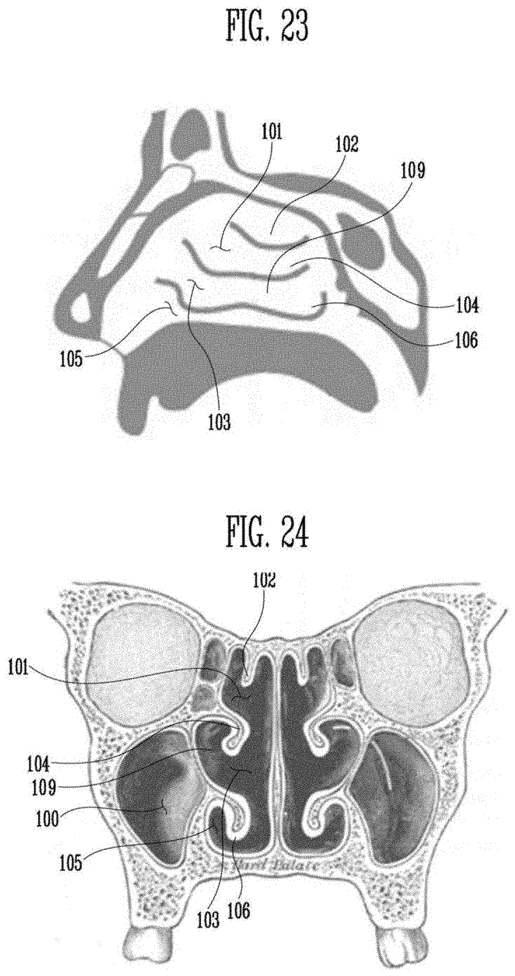

FIG. 20 is a view for describing a maxillary sinus tube which is a part of the apparatus for puncturing a maxillary sinus according to the present invention, FIG. 21 is a view for describing a tube front wing of the maxillary sinus tube, and FIG. 22 is a view for describing inserting and installing the maxillary sinus tube at a punctured part of the posterior fontanel using a tube insertion tool.

As illustrated in FIGS. 20 to 22, a maxillary sinus tube 70 which is a part of the apparatus 1 for puncturing a maxillary sinus includes a tube main body 71, a tube front end 72, a tube front wing 73, a tube hind wing 74, and a tube cover 75. The apparatus 1 for puncturing the maxillary sinus and the maxillary sinus tube are medical appliance.

The tube main body 71 may provide a passage for irrigating or administering drugs into the maxillary sinus 100, may be manufactured in a size and shape corresponding to the hole formed at the posterior fontanel 109, and may include the tube front wing 73 to be described below at a front end thereof to remain inserted in the hole and a tube hind wing 74 to be described below at a rear end thereof. Also, when a large tube main body 71 is required, the size of the hole formed at the posterior fontanel 109 may of course be widened.

To be easily inserted into the hole formed at the posterior fontanel 109, i.e. the punctured portion of the posterior fontanel 109, the tube main body 71 may have the tube front end 72 formed to be gradually thinner toward the front.

The tube front wing 73 is disposed at a front end of the tube main body 71 to allow the maxillary sinus tube 70 to remain inserted in the hole.

To facilitate an insertion and a removal of the tube main body 71, the tube front wing 73 may be manufactured using a material that is bendable while being rigid to a certain degree and may be configured of a plurality of wings.

Specifically, as illustrated in FIG. 21, for each of the plurality of tube front wings 73 to be easily folded when inserting and removing the maxillary sinus tube 70, the plurality of wing portions are prevented from overlapping each other while the plurality of wing portions may be preferably manufactured such that a sum L1+L2+L3+L4 of lengths corresponding to an outer circle (radius R2) forming outer portions of the plurality of wings is smaller than a circumference 2πR1 of an inner circle (radius R1) forming inner portions of the plurality of wings. Also, although the length of R2 is ideal when R2−R1 is smaller than R1, the length of R2 may preferably be formed not to be too small in consideration of the size of the hole formed at the posterior fontanel 109 since the maxillary sinus tube 70 should be at a fixed position in the posterior fontanel 109. Meanwhile, even when R2−R1 is larger than R1, the length of R2 does not becomes a problem when the maxillary sinus tube 70 is inserted or removed since the wing portions gather within the inner circle when the maxillary sinus tube 70 is being removed.

The tube hind wing 74 is disposed at the rear end of the tube main body 71 to allow, together with the tube front wings 73, the maxillary sinus tube 70 to remain inserted in the hole.

The tube hind wing 74 has a size capable of preventing the maxillary sinus tube 70 from being inserted into the maxillary sinus 100.

The tube cover 75 may cover the maxillary sinus tube 70 inserted and installed through the hole formed at the posterior fontanel 109 to block a portion between the maxillary sinus 100 and the middle meatus 103 and may be removed when irrigating the maxillary sinus 100 or administering drugs to the maxillary sinus 100.

A tube insertion tool 80 may be used as illustrated in FIG. 22 to insert and install the maxillary sinus tube 70 at the hole formed at the posterior fontanel 109, i.e. the punctured portion of the posterior fontanel 109.

The tube insertion tool 80 includes a tool main body 81, a tool handle 82, and a support part 83.

A method of inserting the maxillary sinus tube 70 using the tube insertion tool 80 is as follows. When a force is applied to the tool handle 82 while the tool main body 81 is fitted in the tube main body 71 of the maxillary sinus tube 70, the maxillary sinus tube 70 is inserted into the punctured portion as the force is transmitted to the tube hind wing 74 by the support part 83. Then, the tube insertion tool 80 is removed from the maxillary sinus tube 70 and stored.

The maxillary sinus tube 70 inserted and installed at the punctured portion of the posterior fontanel 109 as above has to be removed when irrigating the maxillary sinus 100 or administering drugs into the maxillary sinus 100 is not required. The maxillary sinus tube 70 may be removed by holding the tube main body 71 using forceps and applying a force toward the nasal septum (the center).

As described above, the maxillary sinus tube 70 may be inserted and installed through the hole formed at the posterior fontanel 109 after the irrigation tube 60 is removed in order to perform irrigation or drug administration for several times as needed. In addition, since the maxillary sinus 100 may be a place in which drugs are stored due to its wide hollow space, the maxillary sinus 100 might be used in the future as a drug storage place not only when treating maxillary sinusitis but also when drugs need to be continuously administered for a long period of time due to other diseases. For this, when drugs are injected into the maxillary sinus 100 and the maxillary sinus tube 70 is blocked by the tube cover 75 while the maxillary sinus tube 70 is installed at the hole formed at the posterior fontanel 109, the drugs injected into and stored in the maxillary sinus 100 are continuously supplied to the nasal cavities through the maxillary sinus ostium and are supplied to a stomach via a nasopharynx, a pharynx, and an esophagus.

As described above, in the present invention, difficulty of a surgical procedure may be lowered and a danger may be reduced due to not puncturing the bones 107, and the posterior fontanel 109 without the bones 107 may be easily detected using ultrasonic waves and punctured, thereby promoting the operator's convenience.

Although the present invention has been described by mainly describing the embodiments of the present invention, the embodiments are merely examples and are not intended to limit the present invention. Those of ordinary skill in the art to which the present invention pertains will understand that various combinations, modifications, or applications not exemplified in the embodiments are possible without departing from the technical contents of the embodiments of the present invention. Consequently, technical contents related to modifications and applications that may be easily devised from the embodiments of the present invention should be construed as being included in the present invention.

1. An apparatus for puncturing a maxillary sinus, the apparatus comprising:
a main body gripped by an operator and having a puncturing tube;
an ultrasonic probing unit disposed in the main body and configured to detect a posterior fontanel without bones along a direction from a middle meatus to a maxillary sinus;
a puncturing unit disposed in the main body, and having a needle disposed at a front end of the puncturing tube and configured to cauterize the posterior fontanel using electricity and puncture the posterior fontanel and a cautery wire configured to pass through the puncturing tube and supply electricity to the needle;
an operation unit disposed in the main body and configured to move the puncturing unit toward the posterior fontanel; and
an irrigation tube disposed to be fitted to the cautery wire in the puncturing tube and installed at the posterior fontanel punctured by the needle.

2. The apparatus according to claim 1, wherein the ultrasonic probing unit comprises:
an ultrasonic main body disposed at a rear portion of the main body;
an ultrasonic probe disposed at a front portion of the main body; and
a coaxial lead wire configured to transmit an electrical signal from the ultrasonic main body to the ultrasonic probe.

3. The apparatus according to claim 2, wherein:
the main body further comprises a probing tube in a lengthy form to be inserted into the middle meatus and having the ultrasonic probe disposed at a front end thereof and the coaxial lead wire passing through an inner portion thereof; and
the puncturing tube is in a lengthy form to be inserted into the middle meatus and parallel to the probing tube.

4. The apparatus according to claim 3, wherein the operation unit further comprises a mover to move the cautery wire along the puncturing tube.

5. The apparatus according to claim 4, wherein the mover further comprises:
a moving block configured to move while fixing the cautery wire to allow the cautery wire to move along the inner portion of the puncturing tube;
a rail disposed in the main body and configured to guide a movement of the moving block; and
a trigger configured to move the moving block on the rail in accordance with an operator's operation.

6. The apparatus according to claim 4, wherein the mover further comprises:
a pair of rollers configured to rotate while having the intervening cautery wire to allow the cautery wire to move along the inner portion of the puncturing tube;
a rack directly or indirectly connected to the pair of rollers and configured to realize the rotation of the pair of rollers; and
a trigger configured to move the rack in accordance with the operator's operation to allow the pair of rollers to rotate.

7. The apparatus according to claim 3, further comprising a rotation unit disposed in the main body and configured to allow the front end of the puncturing tube to be bent.

8. The apparatus according to claim 7, further comprising a sheath configured to hold portions of the puncturing tube besides the front end thereof being bent,
wherein a portion of the sheath pointing toward the posterior fontanel which is a direction in which the front end of the puncturing tube is bent is relatively thick, and other remaining portions thereof are relatively thin.

9. The apparatus according to claim 8, wherein the sheath comprises:
a guide structure configured to form a front end of the sheath and have a convex outer shape and a rounded inner shape;
a sheath space configured to provide a place in which the front end of the puncturing tube may be bent and have a space thereof secured by the guide structure; and
a sheath opening configured to provide a passage through which the needle guided by the guide structure passes.

10. The apparatus according to claim 3, further comprising an inlet portion disposed at the rear end of the puncturing tube and configured to have a cross-sectional area gradually enlarging toward the rear and form an inlet of the puncturing tube for introducing the cautery wire.

11. The apparatus according to claim 1, further comprising a notification unit configured to inform the operator of a value detected by the ultrasonic probing unit.

12. A medical appliance comprising:
the apparatus according to claim 1 and
a maxillary sinus tube inserted and installed at a punctured portion of the posterior fontanel punctured by the apparatus to irrigate the maxillary sinus or administer drugs into the maxillary sinus.
